(19) United States
(12) Patent Application Publication
(10) Pub. No.: US 2011/0131697 A1
(43) Pub. Date: Jun. 9, 2011

(54) POSTURE-IMPROVING GARMENT

(75) Inventor: Takemasa Kawahara, Osaka (JP)

(73) Assignee: Sea Shell Co., Ltd.

(21) Appl. No.: 12/737,725

(22) PCT Filed: Aug. 7, 2009
(86) PCT No.: PCT/JP2009/064054

§ 371 Date: Feb. 10, 2011

(30) Foreign Application Priority Data
Aug. 11, 2008 (JP) 2008-207043

(51) Int. Cl. A61F 5/02; A61F 5/01

(52) U.S. Cl. 2/44

(57) ABSTRACT

Improved posture-improving garment is disclosed. The improved posture-improving garment has a hard-to-stretch region provided in a rear body part, the hard-to-stretch region having higher inelasticity than a garment main part, wherein a region woven in a portion corresponding to a vicinity of a lower part of a rhomboid muscle, and a region woven in a portion corresponding to lower fibers of a trapezius muscle and a vicinity of an inferior posterior serratus muscle, are woven in an integrated fashion with the hard-to-stretch region.

### TECHNICAL FIELD

The present invention relates to a posture-improving garment which is capable of improving a rounded back (stooped posture) by promoting expansion of the chest and extension of the thoracic spine by guiding the scapulae downwards.

### BACKGROUND ART

A rounded back not only detracts from a person's physical appearance, but also is a cause of various ailments, such as lower back pain, stiff shoulders, and the like, and therefore corrective underwear, and the like, aimed at improving posture has been commercially available in the prior art. However, corrective underwear, and the like, is highly constrictive and impedes mobility, and is therefore not suitable for prolonged use on a daily basis or use in situations involving movement of the body, such as sports. In this respect, in recent years, inner wear and sports wear which applies taping theory and seeks to correct a rounded back by supporting the movement of the muscles and joints has been proposed.

For example, an under shirt has been proposed in which constricting portions are formed in the shoulder lines, armholes and sides on the front body area of an under shirt, and in the upper part of the rear body area which is linked by the shoulder lines and sides on the front body area (see Patent Document 1).

Furthermore, a posture correcting garment has been proposed as a garment for correcting upper body posture, in which band-shaped tensioning portions having greater constricting force than other portions are provided on the upper part of the rear face in substantially parallel leftward and rightward directions, both ends of the band-shaped tensioning portions are linked to tensioning portions curved along the armholes on the front face, and a tensioning portion linking the two tensioning portions is formed in the central area of the upper part of the rear face (see Patent Document 2).

### [Patent Document 1] Japanese Laid-Open Patent Application 2005-248391

### [Patent Document 2] Japanese Laid-Open Patent Application 2006-320640

### DISCLOSURE OF THE INVENTION

### Problems to be Solved by the Invention

A brief description of a rounded back posture is now given. A rounded back means a posture in which the acromia are displaced forwards and the back is curved. A rounded back occurs because the scapulae rotate outwards, mainly due to weakening of the antigravity muscles in the back, causing the acromia to be displaced forward. Here, the antigravity muscles are the rhomboid muscles, the trapezius muscle, and the inferior posterior serratus muscle. Furthermore, the thoracic spine curves severely backwards from the correct position and the chest falls forwards and is constricted. Therefore, the back appears to be curved, the chest narrows and the head and shoulders are displaced forwards, causing the weight of the head to place an excessive load on the upper part of the trapezius muscle and thus leading to stiffness in the shoulders, as well as producing lower back pain due to the weight of the trunk bearing down on the lumbar area.

In the underwear described in Patent Document 1 and the attitude correcting garment described in Patent Document 2, constricting portions (tensioning portions) which are formed in the upper body area so as to intersect diagonally in a cross shape on the back and pass around both shoulders forcibly guide the acromia backwards so as to correct outward rotation of the scapulae, forward movement of the caput humeri and clavicle, and severe backward curvature of the thoracic spine, which cause a rounded back, as a result of which the wearer's scapulae are caused to rotate horizontally inwards and the caput humeri and clavicle are forcefully pulled backwards, promoting expansion of the chest and extension of the thoracic spine and causing the chest to project outwards. Therefore, upon external observation, it appears as if the rounded back has been corrected.

However, the forced backward pulling of the caput humeri 22 and the clavicle 23 and horizontal inward rotation of the scapulae 21 produced by the above-mentioned external constrictive forces are not based on correct functionally coordinated movement of the bones in the scapular region (see FIGS. 7A and 7B) . More specifically, when the acromia are forcibly guided backwards, the scapulae 21 rotate horizontally inwards, giving rise to shoulder pain due to the load placed on the shoulder joints between the caput humeri 22 and the scapulae 21, and the wearer's chest is forcibly projected outwards by forward elevation of the sternum, causing lower back pain due to the load placed on the lumbar region. Hence, there is a problem in that, when the underwear or posture correcting garment described in the Patent Documents is worn, although it appears as if the posture has been corrected, there is conversely an increased risk of fatigue and joint complaints in the wearer.

There is also a problem in that, if the chest is forcibly projected outwards, then the diaphragm is pressed upwards due to the unnecessary forward elevation of the sternum, thus inhibiting the opening and closing movement of the sternum and the action of the diaphragm and making abdominal breathing more difficult (see FIG. 8) . Abdominal breathing is generally regarded to have benefits in terms of tranquilizing effects, dietary effects, relief of lower back pain, and the like, compared to thoracic breathing, but the above-mentioned benefits cannot be expected when wearing the underwear or attitude correcting garment described in the Patent Documents, which hinder abdominal breathing.

Moreover, a radical solution has not been provided for the weakening of the antigravity muscles in the back, which is the main cause of a rounded back.

In this respect, in order to improve a rounded back reasonably by promoting correct functionally coordinated movement of the bones from the scapulae, it is essential to promote muscular action of the antigravity muscles in the back and guide the scapulae downwards. More specifically, if the antigravity muscles perform action, then the scapulae slide downwards rather than rotating inwards, and in conjunction with this, the acromia are guided reasonably to a correct lower posterior position, in addition to which the sternum and the ribcage are elevated due to slight backward movement and backward rotation of the clavicle, the chest is expanded reasonably, and at the same time, extension of the thoracic spine is promoted. As a result of this coordinated movement of the bones, it is possible to improve posture without applying load to the shoulder joints between the caput humeri and the scapulae, or to the lumbar region, and without pressing the diaphragm upwards. An object of the present invention is to provide a posture-improving garment which improves a rounded back by promoting muscular action of antigravity muscles in the back, without applying a load to the wearer's body, through a correct coordinated movement of bones from the scapulae.

### Means Used to Solve the Above-Mentioned Problems

In order to resolve the above-mentioned problem, the present invention was configured as follows. In other words, the posture-improving garment relating to the present invention is a posture-improving garment wherein a hard-to-stretch region having higher inelasticity than a garment main part is provided in a rear body part, characterized in that the hard-to-stretch region is formed by a region woven in a portion corresponding to a vicinity of a lower part of a rhomboid muscle when the garment is worn and having tension respectively acting diagonally inwards and downwards, and a region woven in a portion corresponding to lower fibers of the trapezius muscle and the vicinity of the inferior posterior serratus muscle and having a tension acting downwards.

Furthermore, the posture-improving garment relating to the present invention is a posture-improving garment, wherein a hard-to-stretch region having higher inelasticity than a garment main part is provided in a rear body part, characterized in that a region woven in a portion corresponding to a vicinity of a lower part of a rhomboid muscle when the garment is worn, and a region woven in a portion corresponding to lower fibers of the trapezius muscle and the vicinity of the inferior posterior serratus muscle, are woven in an integrated fashion in the hard-to-stretch region; the region woven in the portion corresponding to the vicinity of the lower part of the rhomboid muscle being woven in a laterally symmetrical shape comprising two triangular shapes aligned in a horizontal direction which meet only at vertices of lower edges on the central longitudinal axis of the garment main part, each of which triangular shapes having a tension acting diagonally inwards and downwards; and the region woven in the portion corresponding to the lower fibers of the trapezius muscle and the vicinity of the interior posterior serratus muscle being woven in an inverted trapezoidal shape or an inverted triangular shape which meets the lower edges of the triangular shapes and has a height extending from the lower edges to the lower portion of the rear part of the garment, and having a tension acting downwards.

### Effect of the Invention

If a posture-improving garment according to the present invention is worn, it is possible to improve a rounded back reasonably, without applying a load to the wearer's body, and without giving rise to shoulder pain caused by inward rotation of the scapulae due to forced backward guiding of the acromia, lower back pain caused by forced forward projection of the chest, or inhibition of abdominal breathing, and the like.

### Best Mode for Carrying Out the Invention

Below, a best mode for carrying out a posture-improving garment according to the present invention is described below on the basis of embodiments.

The posture-improving garment according to the present invention includes various garments which are fitted tightly to a wearer's body, such as inner wear, sports wear, rash guards, “mini T-shirts, ” and the like, as well as other items which are fitted tightly to a wearer's body, such as swimwear, wet suits, and the like. Sports wear is used in the specific descriptions of the first to third embodiments given below.

Firstly, a posture-improving garment 1 according to a first embodiment will be described. FIG. 1 is a diagram showing a rear body region of a posture-improving garment 1 relating to the present invention. The posture-improving garment 1 is constituted by a garment main part 2, and hard-to-stretch regions, namely, regions 3 (hereinafter, first hard-to-stretch regions 3) woven into portions of the garment corresponding to the vicinity of the lower part of the rhomboid muscles 11 and having a tension acting diagonally inwards and downwards, and a region 4 (hereinafter, second hard-to-stretch region 4) woven into a portion of the garment corresponding to the lower fibers of the trapezius muscle 12 and the vicinity of the inferior posterior serratus muscle 13, and having a tension acting downwards. The first hard-to-stretch regions 3 and the second hard-to-stretch region 4 are described separately in terms of their action on the muscles of the respective areas, but in actual practice these regions are woven in an integrated fashion and act together as a single region.

The garment main part 2 is woven from a stretchable fabric having stretchability similar to that of generally available sportswear, and the like, and the first hard-to-stretch regions 3 and the second hard-to-stretch region 4 are woven from a stretchable fabric which is harder to stretch than the garment main part 2. The first hard-to-stretch regions 3 are woven in two laterally symmetrical right-angled triangle shapes which meet only at the vertices of the lower edges on the central longitudinal axis in the vicinity of the center of the rear part of the garment, and the second hard-to-stretch region is woven in an inverted trapezoidal shape which meets the lower edges of the two right-angled triangle shapes and has a height extending from these lower edges to the lower portion of the rear part of the garment. The second hard-to-stretch region 4 may be woven in an inverted triangle shape. The first hard-to-stretch regions 3 and the second hard-to-stretch region 4 are woven as a single integrated hard-to-stretch region which excludes the dotted lines shown in FIG. 1. Furthermore, provided that prescribed stretching forces are obtained respectively, it is also possible to form the hard-to-stretch regions by using a different material, by adopting a different weaving method, or by bonding or thermally printing a fabric that is harder to stretch than the garment main part 2, onto the garment main part 2.

In this way, hard-to-stretch regions are woven principally in the central portion and lower portion of the rear part of the garment, and when the garment is worn and fitted tightly to the wearer, the first hard-to-stretch regions 3 and the second hard-to-stretch region 4 are pulled downwards, and therefore downward tensions in the direction of the arrows A are produced by the hard-to-stretch regions.

FIG. 2 is a diagram showing the correspondence between the weaving positions of the first hard-to-stretch regions 3 and the second hard-to-stretch region 4, and the muscles, when the above-mentioned posture-improving garment 1 is worn. As shown in FIG. 2A, the first hard-to-stretch regions 3 are woven at positions which cover the vicinity of the lower part of the rhomboid muscles 11. The rhomboid muscles 11 perform muscular action to slide the scapulae 21 diagonally inwards and upwards by traveling diagonally outwards and downwards from the sixth to fourth cervical vertebrae to the inner edge of the scapulae 21, but the first hard-to-stretch regions 3 which are woven at positions corresponding to the lower part of the rhomboid muscles 11 have a tension acting inwards and downwards when worn due to being woven in right-angled triangle shapes of which the upper and lower edges converge toward the central longitudinal axis of the garment main part 2, and cause a tension to act diagonally inwards and downwards in the direction of arrows B so as to promote inward movement of the rhomboid muscles 11 without causing inward rotation relative to the scapulae 21.

Furthermore, as shown in FIG. 2B, the second hard-to-stretch region 4 is woven in a position covering the lower fibers of the trapezius muscle 12 and the vicinity of the inferior posterior serratus muscle 13. The lower fibers of the trapezius muscle 12 travel downwards and perform muscular action to pull the scapulae 21 downwards, and the inferior posterior serratus muscle 13 is positioned in the deep layer of the lower fibers of the trapezius muscle 12 and performs muscular action to restrict the lower fibers of the trapezius muscle 12 from reflexing backwards. The second hard-to-stretch region 4 which is woven in an inverted trapezoidal shape has a tension acting downwards in the direction of arrows C when the garment is worn, and promotes muscular action of the lower fibers of the trapezius muscle 12 to guide the scapulae 21 downwards, as well as promoting muscular action of the inferior posterior serratus muscle 13 to restrict the lower fibers of the trapezius muscle 12 from reflexing backwards due to the downward guiding of the scapulae 21 by the lower fibers of the trapezius muscle 12, and to assist the muscular action of the lower fibers of the trapezius muscle 12.

As shown in FIG. 2C, since the first hard-to-stretch regions 3 and the second hard-to-stretch region 4 act simultaneously and conjointly on the respective muscular actions, then the direction of the tension which guides the scapulae 21 downwards is the direction of the arrows A, which is sum of the vectors of the arrows B and arrows C described above, and muscular action of the rhomboid muscles 11 and the lower fibers of the trapezius muscle 12 is promoted to guide the scapulae 21 downwards while causing the scapulae 21 to slide inwards and downwards. Therefore, the scapulae 21 do not rotate inwards, and the direction of movement of the scapulae 21 is as indicated by the arrows D.

The above-mentioned downward guiding of the scapulae 21 is described specifically below with reference to FIG. 3 and FIG. 4. FIG. 3 is a rear side diagram showing downward guiding of the scapulae 21, and FIG. 4 is a right-hand side diagram showing coordinated bone movement after downward guiding of the scapulae 21.

Firstly, the tensions acting when the posture-improving garment 1 is worn and the downward guiding of the left and right scapulae 21 is described with reference to FIG. 3. The first hard-to-stretch regions 3 promote inward movement of the rhomboid muscles 11 by causing tensions to act inwards and downwards from the inner edges of the scapulae 21, thereby causing the scapulae 21 to slide diagonally inwards and downwards. Simultaneously, the second hard-to-stretch region 4, which is woven in a position that also covers the lower part of the fossa infraspinata of the scapulae 21, promotes muscular action of the lower fibers of the trapezius muscle 12 and of the inferior posterior serratus muscle 13, due to the downward tension, as well as guiding the left and right scapulae 21 downwards. Therefore, the direction of action of the first hard-to-stretch regions 3 and the second hard-to-stretch region 4 is that indicated by the arrows A, and the direction of movement of the scapulae 21 is as indicated by the arrow D.

Next, the coordinated movement of the bones after the above-mentioned downward guiding of the scapulae 21 is described with reference to FIG. 4. When the posture-improving garment 1 is worn, the caput humeri 22 are also pulled backwards and downwards in conjunction with the downward guiding of the scapulae 21. In this case, since the scapulae 21 are guided so as to slide downwards without being forced to rotate horizontally inwards, then the caput humeri 22 are pulled backwards and downwards to a correct position. The backward pulling of the caput humeri 22 does not cause the acromia to be displaced backwards, as in the prior art. Furthermore, as the clavicle 23 is rotated backwards while being pulled slightly backwards in conjunction with the downward guiding of the scapulae 21, the sternum 24 and ribcage 25 are guided so as to elevate, without the chest being forced to project forward, and consequently, the chest is expanded while simultaneously promoting extension of the thoracic spine 26. Moreover, since the chest is not elevated in a forward direction, the diaphragm and the chest are not constricted and abdominal breathing is not inhibited.

If a posture-improving garment 1 of this kind is worn, it is possible to improve a rounded back reasonably, without applying a load to the wearer's body, and without giving rise to shoulder pain caused by forced inward rotation of the scapulae 21, lower back pain caused by forced forward projection of the chest, or inhibition of abdominal breathing, and the like.

Next, a posture-improving garment 1 relating to a second embodiment is described with reference to FIG. 5. Compared to the posture-improving garment 1 of the first embodiment described above, this posture-improving garment 1 is characterized in that the second hard-to-stretch region 4 is woven below a line linking the inferior angles of the left and right scapulae 21. This second hard-to-stretch region 4 acts on the muscular action of the lower fibers of the trapezius muscle 12 below the inferior angles of the scapulae 21, and guides the scapulae 21 downwards by applying a downward tension when the garment is worn. In order to guide the scapulae 21 downwards, the second hard-to-stretch region 4 should act at least on the lower fibers of the trapezius muscle 12 below the inferior angles of the scapulae 21. The action of the first hard-to-stretch regions 3 and the second hard-to-stretch region 4 on the position corresponding to the inferior posterior serratus muscle 13 is similar to the posture-improving garment relating to the first embodiment described above. Furthermore, weaving the second hard-to-stretch region 4 up to a position covering the lower part of the fossa infraspinata of the scapulae 21 as in the second hard-to-stretch region 4 of the first embodiment achieves stronger downward tensions in the direction of the arrows A.

Next, a posture-improving garment 1 relating to a third embodiment is described with reference to FIG. 6. This posture-improving garment 1 is characterized in that, in addition to the composition of the posture-improving garment 1 according to the first embodiment described above, third hard-to-stretch regions 5 are woven in positions covering the left and right rotator cuffs 14. The rotator cuffs 14 are muscles made up of the subscapularis muscle, the supraspinatus muscle, the infraspinatus muscle and the teres minor muscle, and serve to link the shoulder joint and the humerus and stabilize the scapulae in a correct position. It should be noted that the subscapularis muscle is not illustrated. The lower edges of the third hard-to-stretch regions 5 meet the second hard-to-stretch region 4, and similarly to the second hard-to-stretch region 4, have a downward tension when the garment is worn, thereby guiding both the scapulae 21 and the caput humeri 22 downwards. Therefore, the scapulae 21 can be guided downwards more strongly than with the posture-improving garment 1 relating to the first embodiment described above. In the posture-improving garment 1 according to the third embodiment, the first hard-to-stretch regions 3, the second hard-to-stretch region 4 and the third hard-to-stretch regions 5 are woven as a single integrated hard-to-stretch region which excludes the dotted lines shown in FIG. 6. The third hard-to-stretch regions 5 are woven from a stretchable fabric having greater resistance to stretching than the main part 2, similarly to the first hard-to-stretch regions 3 and the second hard-to-stretch region 4. Furthermore, the actions of the first hard-to-stretch regions 3 and the second hard-to-stretch region 4 in this posture-improving garment 1 are similar to those of the first embodiment described above.

In the embodiments described above, first hard-to-stretch regions 3 are woven in right-angled triangle shapes, as shown in FIG. 1 etc., but the first hard-to-stretch regions 3 are not limited to being woven in right-angled triangle shapes, provided that they are woven in triangle shapes of which the upper and lower edges converge toward the central longitudinal axis and meet at the central longitudinal axis, and provided that they are woven in an integrated fashion so as to meet the second hard-to-stretch region 4 in order to create tensions acting diagonally inwards and downwards.

### INDUSTRIAL APPLICABILITY

The effect of the present invention can be achieved if the first hard-to-stretch regions 3 and the second hard-to-stretch region 4 of the posture-improving garment 1 according to the embodiments described above are woven into a worn item of any kind which fits tightly to the body.

### BRIEF DESCRIPTION OF THE DRAWINGS

FIG. 1 is a rear view of a posture-improving garment relating to the present invention;

FIG: 2A, 2B and 2C are diagrams depicting a relationship with muscles when the posture-improving garment is worn;

FIG. 3 is a diagram depicting a state where downward guiding of the scapulae is promoted in the first embodiment;

FIG: 4 is a right side view depicting coordinated bone movement;

FIG. 5 is a view depicting a state where downward guiding of the scapulae is promoted in a second embodiment;

FIG. 6 is a view depicting a state where downward guiding of the scapulae is promoted in a third embodiment;

FIGS: 7A and 7B are views depicting a chain of movement of bones in the event of inward rotation of the scapulae; and

FIG. 8 is a view depicting forward elevation of the chest.

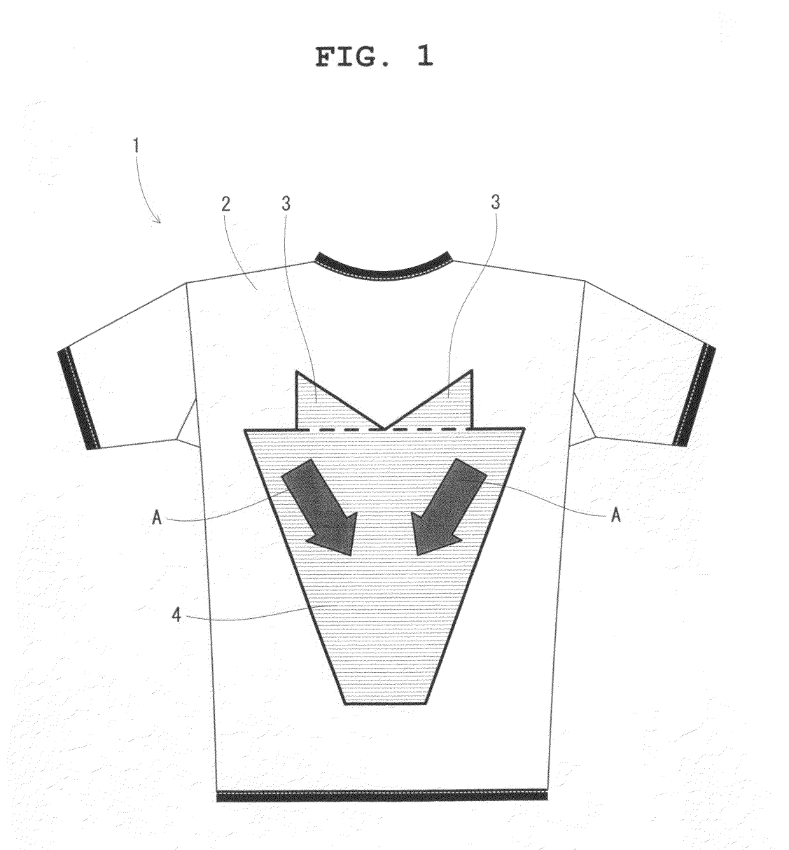

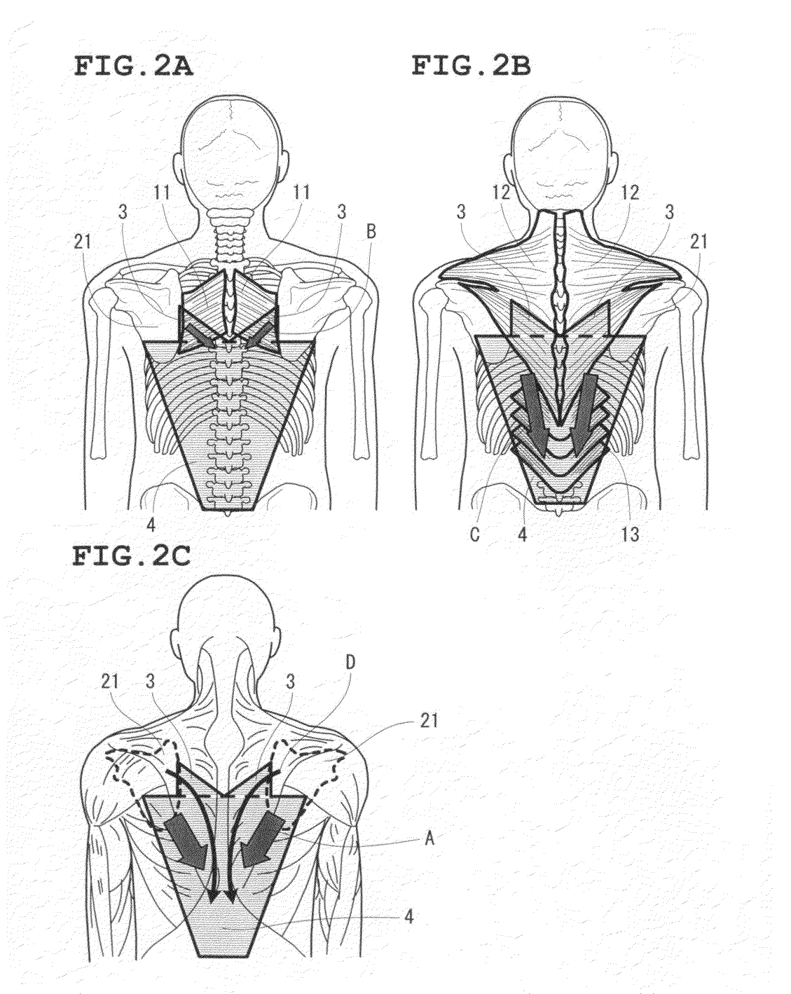

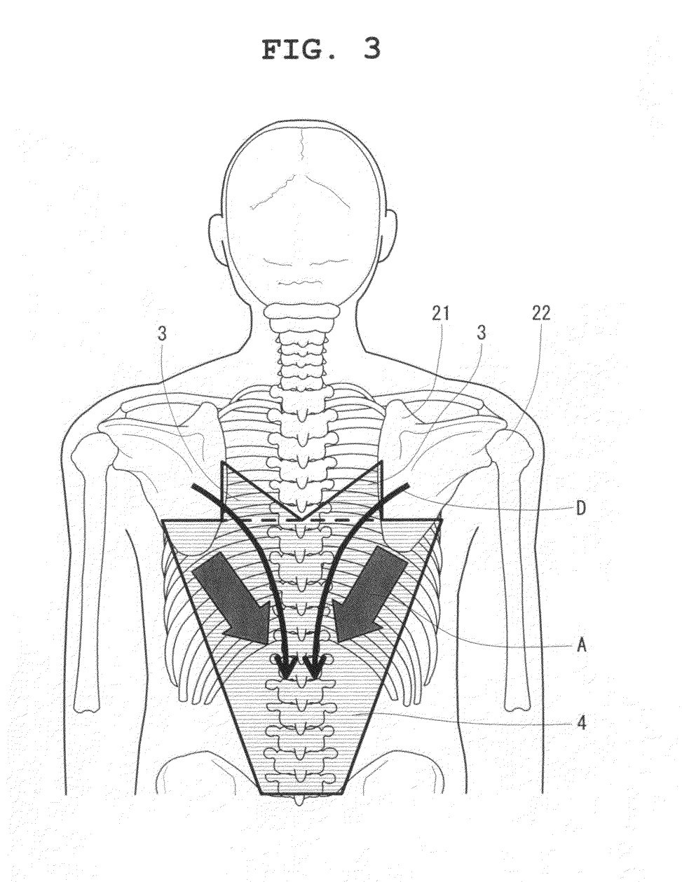

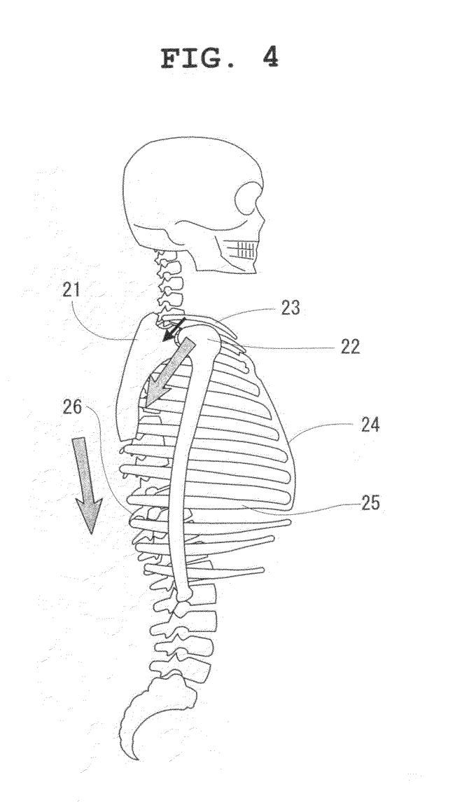

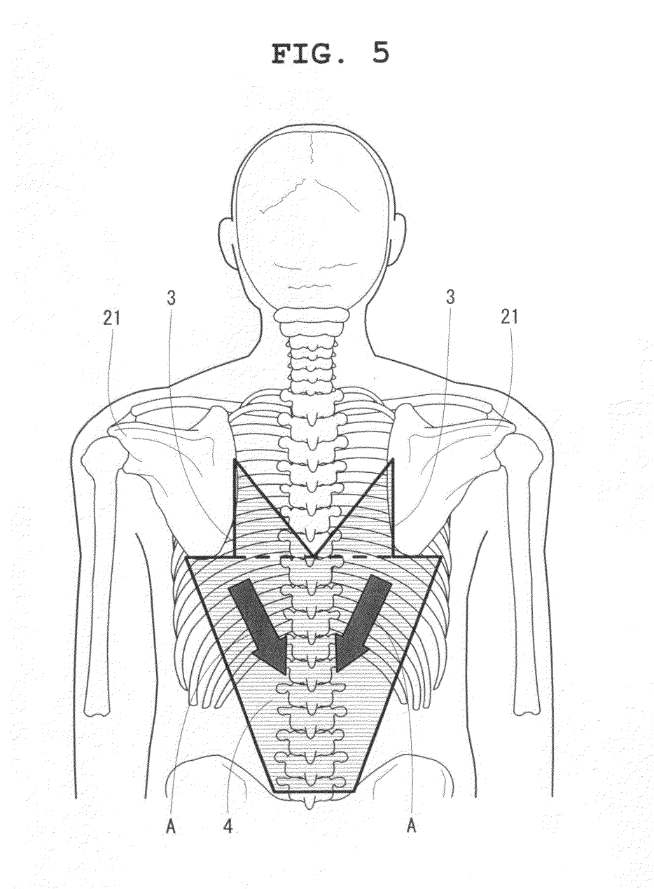

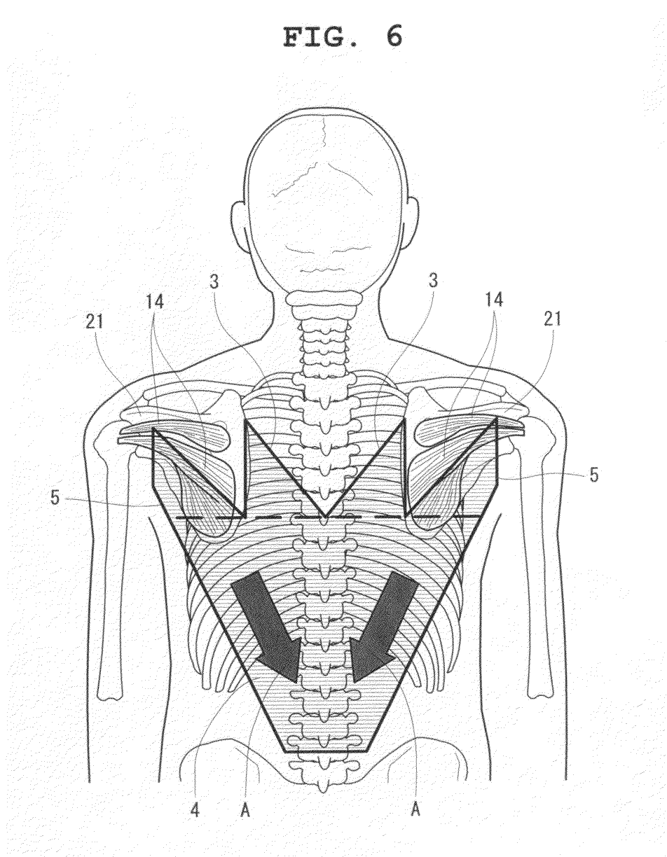

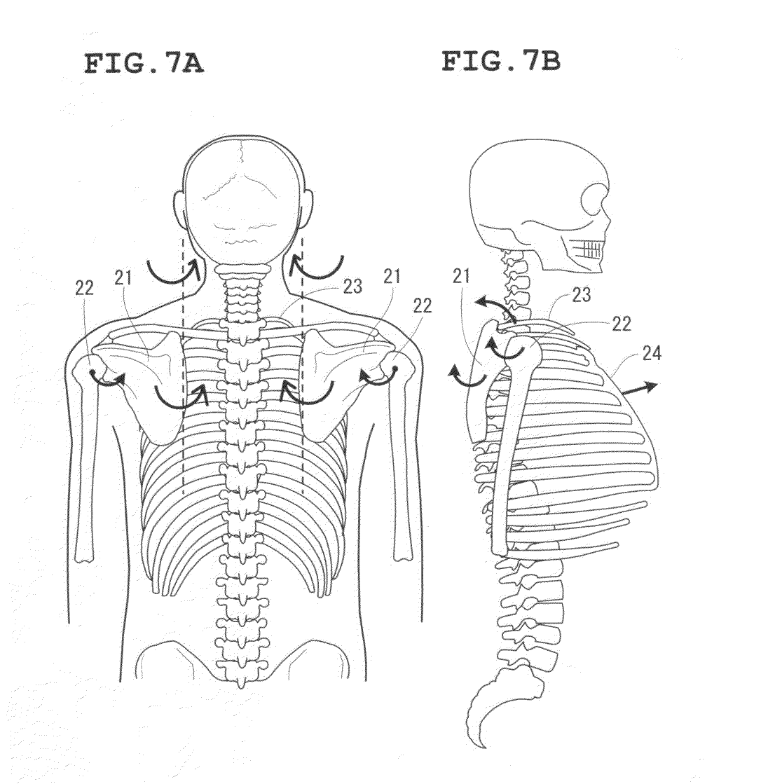

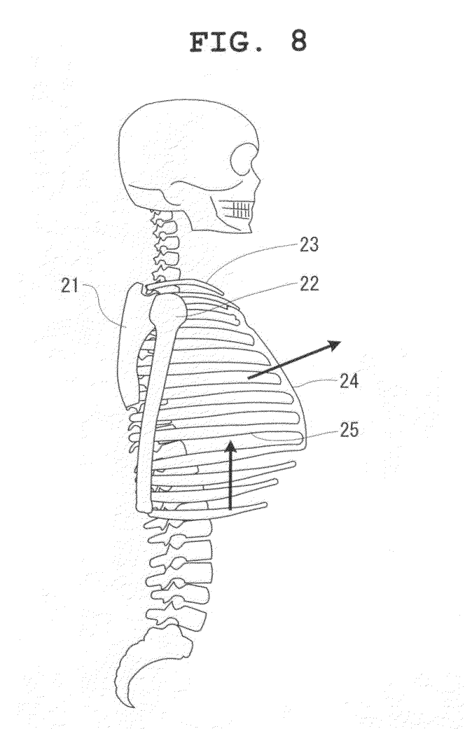

### EXPLANATION OF THE SYMBOLS

1 posture-improving garment

2 garment main part

3 first hard-to-stretch region

4 second hard-to-stretch region

5 third hard-to-stretch region

11 rhomboid muscle

12 trapezius muscle

13 inferior posterior serratus muscle

14 rotator cuff

21 scapula

22 caput humeri

23 clavicle

24 sternum

25 ribcage

26 thoracic spine

1. A posture-improving garment comprising:
a hard-to-stretch region provided in a rear body part, the hard-to-stretch region having higher inelasticity than a garment main part,
wherein:
a region woven in a portion corresponding to a vicinity of a lower part of a rhomboid muscle and having a tension respectively acting diagonally inwards and downwards when worn; and
a region woven in a portion corresponding to lower fibers of a trapezius muscle and a vicinity of an inferior posterior serratus muscle, and having a tension acting downwards when worn,
are woven in an integrated fashion with the hard-to-stretch region.

2. A posture-improving garment comprising:
a hard-to-stretch region provided in a rear body part, the hard-to-stretch region having higher inelasticity than a garment main part, wherein:
a region woven in a portion corresponding to a vicinity of a lower part of a rhomboid muscle when worn; and
a region woven in a portion corresponding to lower fibers of a trapezius muscle and a vicinity of an inferior posterior serratus when worn
are woven in an integrated fashion with the hard-to-stretch region;
the region woven in the portion corresponding to the vicinity of the lower part of the rhomboid muscle being woven in a laterally symmetrical shape having two triangular shapes aligned in a horizontal direction which meet only at vertices of lower edges on a central longitudinal axis of the garment main part, each of which triangular shapes having a tension acting diagonally inwards and downwards; and
the region woven in the portion corresponding to lower fibers of the trapezius muscle and the vicinity of the inferior posterior serratus muscle being woven in an inverted trapezoidal shape or an inverted triangular shape which meets the lower edges of the triangular shapes and has a height extending from the lower edges to the lower portion of the rear body part, and having a tension that acts downwards.
